(19) United States
(12) Patent Application Publication
(10) Pub. No.: US 2023/0414385 A1
(43) Pub. Date: Dec. 28, 2023

(54) IMPLANTABLE SCAFFOLDS HAVING BIODEGRADABLE COMPONENTS AND METHODS OF MANUFACTURING AND USE THEREOF

(71) Applicants: Kamal RAMZIPOOR, Fremont, CA (US); Afsoon FALLAHI, Mountain View, CA (US)

(72) Inventors: Kamal RAMZIPOOR, Fremont, CA (US); Afsoon FALLAHI, Mountain View, CA (US)

(21) Appl. No.: 18/324,477

(22) Filed: May 26, 2023

Related U.S. Application Data
(60) Provisional application No. 63/366,997, filed on Jun. 24, 2022.

(51) Int. Cl. A61F 2/86; A61F 2/958

(52) U.S. Cl. CPC A61F 2/86; A61F 2/958; A61F 2002/91558

(57) ABSTRACT

Various endovascular scaffolds and methods of making and using the endovascular scaffolds are disclosed. In one variation, an endovascular scaffold is disclosed comprising a plurality of undulating rings and a plurality of interconnecting struts connecting the plurality of undulating rings to one another. The plurality of undulating rings can be radially compressible into a delivery configuration and expandable from the delivery configuration to an expanded configuration when deployed. At least some of the interconnecting struts can biodegrade after the endovascular scaffold is deployed within the peripheral vessel.

### CROSS-REFERENCE TO RELATED APPLICATION

This application claims the benefit of priority to U.S. Provisional Application No. 63/366,997 filed on Jun. 24, 2022, the entirety of which is incorporated herein by reference.

### TECHNICAL FIELD

The present disclosure relates generally to the field of stents or scaffolds for treating peripheral vascular disease; more specifically, to implantable endovascular scaffolds having biodegradable components and methods of manufacturing and using such scaffolds.

### BACKGROUND

In recent years there has been growing interest in the use of polymers or polymer-metal composites for use in implantable devices that come into contact with bodily tissues or fluids, particularly blood. However, many polymeric stents or scaffolds have been shown to fail or fracture, similar to their metallic counterparts. Because many polymeric implants such as stents are fabricated through processes such as extrusion or injection molding, such methods typically begin the process by starting with an inherently weak material. In the example of a polymeric stent, the resulting stent may have imprecise geometric tolerances as well as reduced wall thicknesses which may make these stents susceptible to brittle fracture.

A stent or scaffold which is susceptible to brittle fracture is generally undesirable because of its limited ability to collapse for intravascular delivery as well as its limited ability to expand for placement or positioning within a vessel. Moreover, such polymeric stents also exhibit a reduced level of strength. Brittle fracture is particularly problematic in stents as placement of a stent onto a delivery balloon or within a delivery sheath imparts a substantial amount of compressive force in the material comprising the stent. A stent made of a brittle material may crack or have a very limited ability to collapse or expand without failure. Thus, a certain degree of malleability is desirable for a stent or scaffold to expand, deform, and maintain its position securely within the vessel.

More recently, stents or scaffolds made of biodegradable polymers have been proposed to solve the problem of in-stent restenosis. Stents or scaffolds made of biodegradable polymers can be configured to deliver more drugs to a target site than permanent stents. However, such biodegradable stents, when made using traditional fabrication techniques such as extrusion or injection-molding, can exhibit reduced mechanical stability over the life of the stent.

Therefore, an improved biodegradable stent or scaffold is needed that addresses the above shortcomings. Such a biodegradable stent or scaffold should not be susceptible to brittle fracture and should be capable of handling complex loading conditions. Such a biodegradable stent or scaffold should also be biocompatible and amenable to delivery through tortuous and small-diameter vasculature.

### SUMMARY

An endovascular scaffold and methods of making and using the endovascular scaffold are disclosed. In one variation, the endovascular scaffold can comprise a plurality of undulating rings and a plurality of interconnecting struts connecting the plurality of undulating rings to one another. Each of the plurality of interconnecting struts can be positioned between adjacent undulating rings. The plurality of undulating rings can be radially compressible into a delivery configuration and expandable from the delivery configuration to an expanded configuration when deployed.

At least some of the interconnecting struts can be configured to biodegrade over a degradation period after the endovascular scaffold is deployed within the peripheral vessel. The degradation period can be between about 7 months and 24 months. For example, the degradation period can be approximately 18 months.

Moreover, at least one of the plurality of interconnecting struts can comprise growth factors disposed on the interconnecting strut or integrated within a layer of the interconnecting strut. The growth factors can comprise at least one of a vascular endothelial growth factor (VEGF), a platelet-derived growth factor (PDGF), and a heparin-binding EGF-like growth factor (HB-EGF).

More specifically, at least one of the plurality of interconnecting struts can comprise a recessed surface defined along the interconnecting strut. The recessed surface can be shaped or otherwise configured to contain at least some of the growth factors.

At least one of the plurality of interconnecting struts can be made in part of a marine polymer. For example, at least one of the interconnecting struts can be made in part of chitin, chitosan, or a combination thereof.

The plurality of undulating rings can also be made to biodegrade. In these instances, the plurality of undulating rings can be configured to biodegrade at a slower rate than the plurality of interconnecting struts. For example, the plurality of undulating rings can be configured to biodegrade between about 3.0 years and 10.0 years after deployment within the peripheral vessel.

When the endovascular scaffold is deployed within a blood vessel such as a peripheral vessel, the undulating rings supporting the peripheral vessel can be supported in part by an extracellular matrix formed at discontinuities developed in between the undulating rings as the interconnecting struts biodegrade. The discontinuities can refer to empty spaces or gaps in between adjacent undulating rings as the interconnecting struts biodegrade. The discontinuities can also refer to positions along the endovascular scaffold that were formerly occupied by the interconnecting struts. In some variations, when the interconnecting struts biodegrade, the undulating rings can be completely separated from one another.

In some variations, at least one of the plurality of interconnecting struts can be formed by electrospinning. Moreover, at least one of the plurality of interconnecting struts can have a width dimension which is less than a circumference of at least one of the plurality of undulating rings.

At least one of the plurality of interconnecting struts can be comprised of a monofilament. At least one of the plurality of interconnecting struts can be comprised of a plurality of filaments in a multifilament configuration.

The endovascular scaffold can be deployed as part of a balloon-scaffold assembly. The balloon-scaffold assembly can comprise a balloon catheter and the endovascular scaffold crimped onto an inflatable balloon of the balloon catheter.

In some variations, the inflatable balloon can be a semi-compliant balloon. The semi-compliant balloon can be expandable to a minimum diameter of about 2.9 mm at about 6.0 ATMs of pressure. In these and other variations, the semi-compliant balloon can also be expandable to a maximum diameter of about 3.7 mm at about 16.0 ATMs of pressure. The semi-compliant balloon can also be characterized by an upward sloping compliance curve.

Portions of the inflatable balloon can extend through void spaces in between the plurality of undulating rings when the plurality of undulating rings are radially compressed into the delivery configuration. The inflatable balloon can provide structural support to the endovascular scaffold during delivery. The inflatable balloon can be made of an elastic polymeric material. For example, the inflatable balloon can be made in part of polyamide.

In some variations, at least one of the interconnecting struts can be configured to break upon expansion of the endovascular scaffold by the inflatable balloon.

Also disclosed is a method of supporting a peripheral vessel using a balloon-scaffold assembly. The method can comprise introducing a flexible guidewire to a target site within the peripheral vessel, advancing a delivery catheter comprising a balloon-scaffold assembly over the guidewire to the target site, and exposing the balloon-scaffold assembly at the target site. The balloon-scaffold assembly can comprise a balloon catheter and the endovascular scaffold crimped onto an inflatable balloon of the balloon catheter. The inflatable balloon can be a semi-compliant balloon. The endovascular scaffold can be crimped onto the inflatable balloon in a delivery configuration. The method can further comprise inflating the balloon of the balloon catheter to radially expand the endovascular scaffold to an expanded configuration at the target site. The method can also comprise deflating the balloon and withdrawing the delivery catheter and the balloon from the peripheral vessel once the endovascular scaffold provides support for the peripheral vessel in the expanded configuration.

Also disclosed is a method of making an endovascular scaffold for use in a peripheral vessel. The method can comprise dipping a mandrel in a polymeric solution such that a dip-coated substrate forms on the mandrel. The method can also comprise forming a plurality of undulating rings from the dip-coated substrate. The plurality of undulating rings can be radially compressible into a delivery configuration and expandable from the delivery configuration to an expanded configuration (via a balloon catheter) when deployed.

The method can further comprise forming a plurality of interconnecting struts connecting the plurality of undulating rings. For example, the interconnecting struts can be formed using the same pattern forming techniques as the undulating rings (e.g., laser cutting, machining, or other material removal processes). In other variations, the interconnecting struts can be formed by electrospinning filaments connecting adjacent undulating rings.

At least some of the interconnecting struts can be configured to biodegrade over a degradation period when the endovascular scaffold is deployed within the peripheral vessel. The method can further comprise forming a recessed surface along at least one of the plurality of interconnecting struts. The recessed surface can be configured to contain growth factors for inducing formation of an extracellular matrix once the interconnecting struts biodegrades.

The method can further comprise crimping the endovascular scaffold onto an inflatable balloon of a balloon catheter. Crimping the endovascular scaffold onto the inflatable balloon can comprise multiple crimping steps. Each of the crimping steps can be followed by a pause period. The pause period can be between about 1 second to 60 seconds. In some variations, crimping the endovascular scaffold onto the inflatable balloon can comprise between 2 and 30 pause periods.

### BRIEF DESCRIPTION OF THE DRAWINGS

FIG. 1A illustrates a variation of an endovascular scaffold having a plurality of interconnecting struts.

FIG. 1B illustrates the endovascular scaffold of FIG. 1A after the plurality of interconnecting struts have biodegraded.

FIG. 2 illustrates the endovascular scaffold crimped on an inflatable balloon of a balloon catheter.

FIG. 3 illustrates a pressure-diameter plot of a variation of a semi-compliant balloon serving as the inflatable balloon of the balloon catheter.

FIGS. 4A to 4C illustrate example methods of delivering and deploying the endovascular scaffold.

FIG. 5 illustrates an example of a polymeric base substrate that can be used to form components of the endovascular scaffold.

FIG. 6 illustrates an example of a multi-layered polymeric base substrate that can be used to form the endovascular scaffold. The multi-layered polymeric base substrate can comprise base ring structures positioned or fitted upon the polymeric base substrate in order to form undulating rings that can degrade at a slower rate than the interconnecting struts connecting the undulating rings.

FIG. 7 illustrates another example of a multi-layered polymeric base substrate that can be used to form the endovascular scaffold. The multi-layered polymeric base can comprise an additional layer overlaid atop the base ring structures and the polymeric base substrate.

FIGS. 8A-8C illustrate various cross-sections of interconnecting struts made of one or more filaments.

FIG. 9 illustrates a stress-strain plot of polylactic acid (PLLA) at differing molecular weights and their corresponding stress-strain values indicating brittle fracture to ductile failure.

FIG. 10A illustrates an example of a dip-coating machine that can be used to form the polymeric base substrate on a mandrel.

FIGS. 10B and 10C illustrate additional examples of a dip-coating machine comprising one or more articulatable linkages that can adjust a dipping direction of the mandrel.

FIGS. 11A to 11B illustrate partial cross-sectional side views of a variation of a multi-layered polymeric base substrate formed along a mandrel.

FIG. 11C illustrates a cross-sectional end view of the multi-layered polymeric base substrate shown in FIG. 11A.

FIG. 12 illustrates an example of an electrospinning assembly that can be used to form the interconnecting struts of the endovascular scaffold.

### DETAILED DESCRIPTION

The apparatus, devices, systems, and methods described herein are best understood from the detailed description when read in conjunction with the accompanying drawings. It is emphasized that, according to common practice, the various features of the drawings may not be to scale. The dimensions of certain features have been expanded or reduced for clarity and not all features may be visible or labeled in every drawing. The drawings are taken for illustrative purposes only and are not intended to define or limit the scope of the claims to that which is shown.

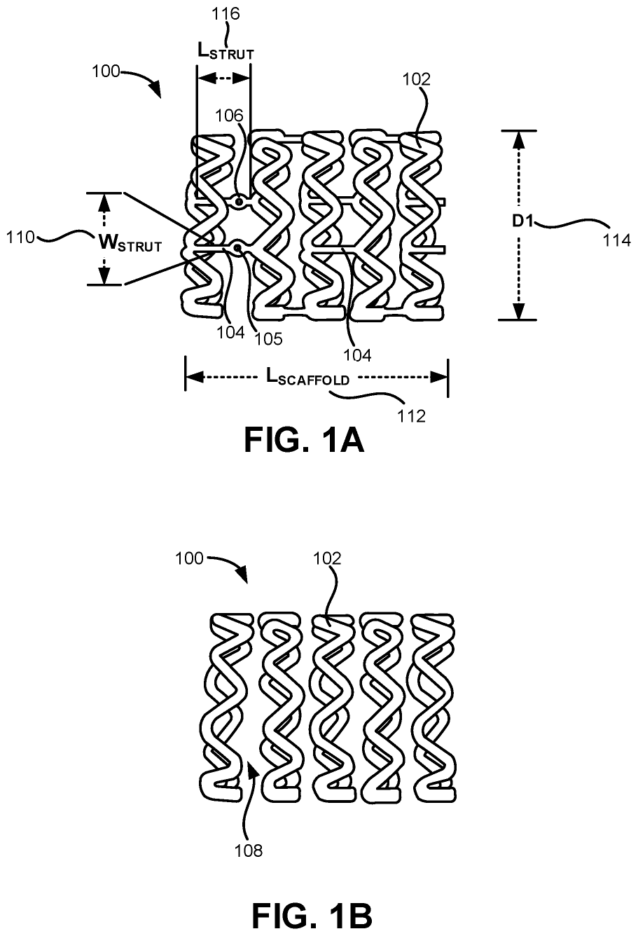

FIG. 1A illustrates one variation of an endovascular scaffold 100 comprising a plurality of undulating rings 102 connected by a plurality of interconnecting struts 104. The endovascular scaffold 100 can be used in any endovascular procedure for treating a peripheral artery disease, including a peripheral artery disease of the lower extremities. For example, the endovascular scaffold 100 can be designed for use in a peripheral blood vessel.

As shown in FIG. 1A, the scaffold 100 can comprise a plurality of undulating rings 102 and a plurality of interconnecting struts 104 connecting the plurality of undulating rings 102 to one another.

In some variations, multiple interconnecting struts 104 can connect adjacent undulating rings 102. In other variations, only one interconnecting strut 104 can connect adjacent undulating rings 102.

In certain variations, the interconnecting struts 104 can be longitudinally offset from one another such that none of the undulating rings 102 are connected to its two immediately adjacent undulating rings 102 by interconnecting struts 104 at the same location along the circumference of the undulating ring 102. In other variations, two or more of the interconnecting struts 104 can be longitudinally aligned such that at least one of the undulating rings 102 is connected to its two immediately adjacent undulating rings 102 by interconnecting struts 104 at the same location along the circumference of the undulating ring 102.

Each of the plurality of undulating rings 102 can have a sinusoidal or zig-zag pattern along a circumference of the undulating ring. In some variations, an interconnecting strut 104 can connect an apex or peak of one undulating ring 102 with an apex or peak of an adjacent undulating ring 102. In other variations, an interconnecting strut 104 can connect a trough of one undulating ring 102 with a trough of an adjacent undulating ring 102. In additional variations, an interconnecting strut 104 can connect an apex or peak of one undulating ring 102 with a trough of an adjacent undulating ring 102.

In some variations, the plurality of undulating rings 102 can be equally spaced apart such that all of the interconnecting struts 104 connecting the undulating rings 102 are the same length. In other variations, the plurality of undulating rings 102 can be spaced closer to one another along a first portion than along a second portion of the endovascular scaffold 100. In these and other variations, the endovascular scaffold 100 can comprise interconnecting struts 104 of different lengths.

Adjacent undulating rings 102 can be axially and rotationally movable relative to one another via one or more interconnecting struts 104. In some variations, a terminal undulating ring 102 can be relatively more flexible than the remainder of the undulating rings 102.

In some variations, the endovascular scaffold 100 can comprise between [2 and 20] undulating rings 102. For example, in one variation, the endovascular scaffold 100 can comprise between [15 and 20 (e.g., 18)] undulating rings 102 connected by interconnecting struts 104. In other variations, the endovascular scaffold 100 can comprise more than 20 undulating rings 102.

Each of the undulating rings 102 can have a ring thickness of between approximately 40 μm to 50 μm. In other variations, each of the undulating rings 102 can have a ring thickness between approximately 60 μm to 300 μm. The low profile of the endovascular scaffold 100 can minimize the formation of blood thrombi at the implantation site.

The endovascular scaffold 100 can have a scaffold length 112, L_{SCAFFOLD}. The scaffold length 112 can be between approximately 1.0 cm and 20.0 cm. More specifically, the scaffold length 112 can be between approximately 1.0 cm and 120 cm.

In some variations, each of the undulating rings 102 can have a length of between approximately 1.0 mm to 3.0 mm.

Each of the struts 104 can have a strut width 110, W_{STRUT}, and a strut length 116, L_{STRUT}. In one variation, the strut width 110 or width dimension can be less than a circumference of at least one of the plurality of undulating rings 102.

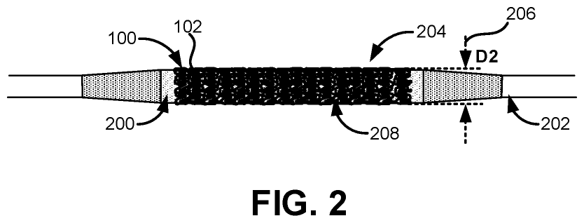

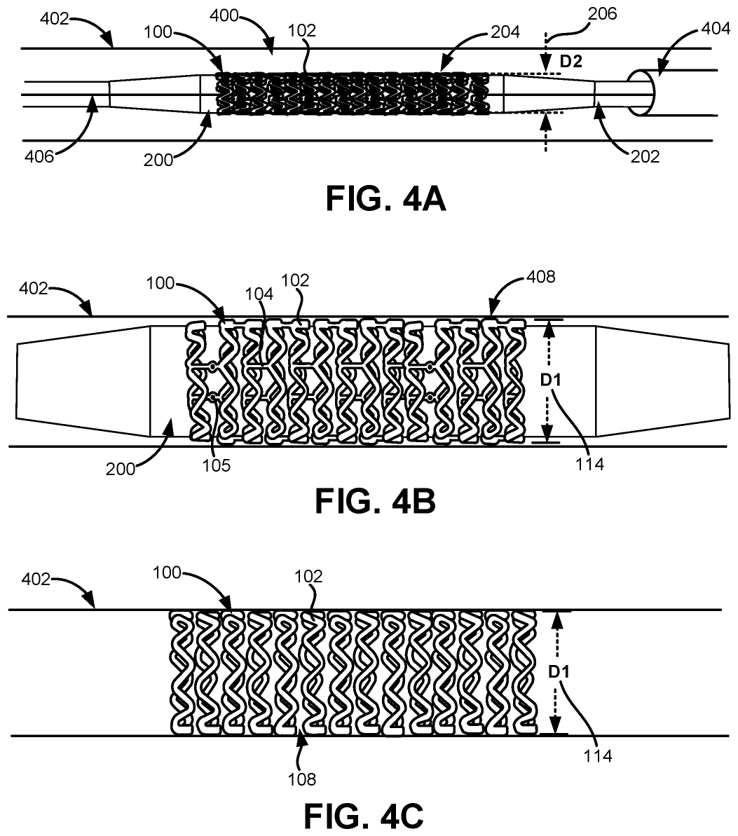

The plurality of undulating rings 102 can be radially compressible from an initial diameter 114 (also shown as D1) into a delivery diameter 206 (see FIGS. 2 and 4A, also shown as D2). The plurality of undulating rings 102 can also be expandable from the delivery diameter 206 back to the initial diameter 114 (see FIG. 4B) when deployed at a target site within a patient.

In some variations, the initial diameter 114 can be between approximately 2.0 times to 9.0 times the size of the delivery diameter 206. In one variation, the initial diameter 114 can be the diameter of a mandrel, cylinder, or tube used to form the undulating rings 102. In other variations, the initial diameter 114 can be the diameter of the endovascular scaffold 100 after one or more rounds of heat treatment or cooling.

The initial diameter 114 can be between approximately 0.7 mm to 2.0 mm. For example, the initial diameter 114 can be approximately 1.2 mm.

The plurality of undulating rings 102 connected by the interconnecting struts 104 can be axially and rotationally moveable or translatable relative to one another. For example, each of the undulating rings 102 can be rotated in a clockwise or counterclockwise direction relative to a neighboring or adjacent ring. Moreover, the endovascular scaffold 100 can be longitudinally compressible or extendible such that the distance between any two of the undulating rings 102 can be increased or decreased based on the applied forces acting on the endovascular scaffold 100.

The endovascular scaffold 100 can be sterilized by radiation such as by gamma sterilization or electron-beam (e-beam) sterilization. The endovascular scaffold 100 can also be sterilized using oxidizing agents such a hydrogen peroxide, ozone, chlorine dioxide, or a combination thereof. The endovascular scaffold 100 can be sterilized using any of the procedures disclosed in U.S. Pat. Nos. 8,309,023, 8,574,493, and 8,858,611, the contents of which are hereby incorporated by reference in their entireties.

In some variations, the interconnecting struts 104 can be made to biodegrade after the scaffold 100 is implanted within a peripheral vessel of a patient such as a superficial femoral artery (SFA).

The interconnecting struts 104 can be made of a biodegradable material. In some variations, the interconnecting struts 104 can be made of a biodegradable polymeric material. In these and other variations, the interconnecting struts 104 can also be made of a biodegradable metallic material or a biodegradable composite material.

In certain variations, the interconnecting struts 104 can be made of a biodegradable polymeric material such as polylactic acid (PLA) or polylactide. More specifically, the interconnecting struts 104 can be made of poly-L-lactic acid (PLLA)/poly-L-lactide, poly-DL-lactic acid (PDLLA)/poly-DL-polylactide, or copolymers, terpolymers, combinations, or mixtures thereof. The molecular weight can range from about 250,000 Daltons (Da) to 3 million Da.

In other variations, the interconnecting struts 104 can be made of poly-glycolic acid (PGA)/poly-glycolide, poly-lactic-co-glycolic acid (PLGA)/poly-lactide-co-glycolide, polycaprolactone such as poly-ε-caprolactone, polydioxanone, polyanhydride, trimethylene carbonate, poly(ß-hydroxybutyrate), poly(g-ethyl glutamate), poly(DTH iminocarbonate), poly(bisphenol A iminocarbonate), poly(ortho ester), polycyanoacrylate, and polyphosphazene, or copolymers, terpolymers, combinations, or mixtures thereof with any of the aforementioned polymers including PLA, PLLA, or PDLLA.

In additional variations, the interconnecting struts 104 can be made of magnesium, a magnesium alloy, or a composite material comprising magnesium and any of the aforementioned biodegradable polymers.

In some variations, the interconnecting struts 104 can be made of a biodegradable marine-based polymer. For example, the interconnecting struts 104 can be made of chitin, chitosan, or a combination thereof. In other variations, the interconnecting struts 104 can be made of a chitosan-based composite material reinforced with polymeric fibers.

The marine-based polymer can be electrospun or dip-coated to create at least part of the interconnecting struts 104. One advantage of making the interconnecting struts 104 out of marine-based polymers is that such polymers can be more easily absorbed by the patient and are loaded with natural anti-inflammatory agents.

In some variations, the interconnecting struts 104 can also be made in part of hyaluronic acid. For example, a hyaluronic acid-based hydrogel can cover or coat at least part of the interconnecting struts 104. The hyaluronic acid-based hydrogel can mimic the mechanical properties of soft tissue and the porosity of the hydrogel can be tuned to modulate the degradation behavior of the interconnecting struts 104.

In certain variations, the interconnecting struts 104 can be made of a biodegradable polymer comprising a polysaccharide such as cellulose or dextran.

In further variations, the interconnecting struts 104 can be made of a modified protein such as fibrin or casein.

The interconnecting struts 104 can be made to biodegrade over/after a degradation period. In some variations, the degradation period can be between approximately 7.0 months and 2.0 years. In other variations, the degradation period can be less than 7.0 months. In further variations, the degradation period can be between approximately 2.0 years and 8.0 years.

When the interconnecting struts 104 are made of a biodegradable polymeric material, the length of the degradation period can depend on factors such as the crystallinity of the polymeric material and/or the molecular weight of the polymeric material. The crystallinity can increase mechanical strength and decrease the degradation time of the struts 104. Struts 104 can be made with biodegradeable polymers having a molecular weight between about 1,000 Da and 100,000 Da.

In certain variations, some of the interconnecting struts 104 can be made of one biodegradable material and other interconnecting struts 104 can be made of another biodegradable material. In further variations, some of the interconnecting struts 104 can be made of a biodegradable material and other interconnecting struts 104 can be made of a non-biodegradable material.

At least some of the interconnecting struts 104 can be configured to completely biodegrade at the end of the degradation period when the scaffold 100 is deployed within the peripheral vessel such that discontinuities 108 (see e.g., FIG. 1B) or empty spaces or gaps are formed in between adjacent undulating rings 102. At this point, the undulating rings 102 can support a vessel wall of the subject without being connected by the interconnecting struts 104. In some variations, when the interconnecting struts 104 biodegrade, the undulating rings 102 can be completely separated from one another.

The undulating rings 102 can also be made of a biodegradable material. In some variations, the undulating rings 102 can be made of a biodegradable polymeric material. In these and other variations, the undulating rings 102 can also be made, in part, of a biodegradable metallic material or a biodegradable composite material.

In certain variations, the undulating rings 102 can be made of a biodegradable polymeric material such as polylactic acid (PLA) or polylactide. More specifically, the undulating rings 102 can be made of poly-L-lactic acid (PLLA)/poly-L-lactide, poly-DL-lactic acid (PDLLA)/poly-DL-polylactide, or copolymers, terpolymers, combinations, or mixtures thereof. The molecular weight of the undulating rings 102 can be higher than the molecular weight of the connecting struts 104.

In other variations, the undulating rings 102 can be made of poly-glycolic acid (PGA)/poly-glycolide, poly-lactic-co-glycolic acid (PLGA)/poly-lactide-co-glycolide, polycaprolactone such as poly-ε-caprolactone, polydioxanone, polyanhydride, trimethylene carbonate, poly(ß-hydroxybutyrate), poly(g-ethyl glutamate), poly(DTH iminocarbonate), poly(bisphenol A iminocarbonate), poly(ortho ester), polycyanoacrylate, and polyphosphazene, or copolymers, terpolymers, combinations, or mixtures thereof with any of the aforementioned polymers including PLA, PLLA, or PDLLA.

In additional variations, at least part of the undulating rings 102 can be made of magnesium, a magnesium alloy, or a composite material comprising magnesium and any of the aforementioned biodegradable polymers.

In some variations, at least part of the undulating rings 102 can be made of a biodegradable marine-based polymer such as chitin or chitosan.

The undulating rings 102 can be configured to biodegrade after/over a degradation period. In some variations, the degradation period for the undulating rings 102 can be between approximately 3.0 years and 7.0 years. In other variations, the degradation period can be less than 3.0 years (especially if the degradation period for the interconnecting struts 104 is less than one year). In further variations, the degradation period can be between approximately 7.0 years and 10.0 years. In further variations, the degradation period can be between approximately 3.0 years and 4.0 years when the degradation period of the interconnecting struts 104 is less than one year. The undulating rings 102 can be made to biodegrade after sufficient blood flow has been restored at the site of a vascular occlusion.

The undulating rings 102 can be made to biodegrade at a slower rate than the interconnecting struts 104. For example, the interconnecting struts 104 can be made to biodegrade three to four times faster than the undulating rings 102. The undulating rings 102 can radially support a target vessel for at least several years after the interconnecting struts 104 have completely biodegraded. Such rapid resorption can result in a scaffold 100 that has a discontinuous structure once the interconnecting struts 104 biodegrade which, in turn, can allow the scaffold 100 to exhibit superior mechincal properties post-implantation, specifically in areas of resistance to fracture under fatigue load and complex stress conditions. In some variations, the risk of restenosis can also be reduced when the scaffold 100 attains its discontinuous structure.

In other variations, the undulating rings 102 and/or at least some of the interconnecting struts 104 can be made of synthetic polymers such as polyethylenes (e.g., polyethylene terephthalate), polycarbonates, polyamides, polysulfones, polyacetals, polyketals, polydimethylsiloxanes, and polyetherketones, polyurethanes, methyl acrylates or methyl acrylic acid, methyl methacrylates, hydroxyethyl acrylates, hydroxyethyl methacrylates, acrylamides such as methacrylamides, glyceryl acrylates, glyceryl methacrylates, vinyls such as styrene, vinyl chloride, pyrrolidone, polyvinyl alcohol, vinyl acetate, propylene, polytetrafluoroethylene, or copolymers, terpolymers, combinations, or mixtures thereof.

In some variations, at least some of the interconnecting struts 104 can comprise at least one elastomeric polymer layer. For example, the interconnecting struts 104 can be formed from or comprise a polymer blend or co-polymer of poly-L-lactide (PLLA) and an elastomeric polymer.

At least one of the interconnecting struts 104 can have a recessed surface 106 defined along a length of the interconnecting strut 104. The recessed surface 106 can be a circular divot or indent defined along the interconnecting strut 104. The recessed surface 106 can be positioned anywhere along the length of the interconnecting strut 104. Additionally, the recessed surface 106 can contain a radiopaque marker within.

The recessed surface 106 of the interconnecting struts 104 can be configured to contain or receive one or more growth factors 105 deposited thereon or integrated therein. In other variations, the growth factors 105 can be integrated into one or more layers (e.g., polymeric layers) of the interconnecting strut(s) 104. The growth factors 105 can be in an amount ranging from 1 nanogram (ng) to 10 miligrams (mg).

The growth factors 105 can comprise at least one of a vascular endothelial growth factor (VEGF), a platelet-derived growth factor (PDGF), and a heparin-binding EGF-like growth factor (HB-EGF).

In some variations, the growth factors 105 can be spray-coated or injected onto the recessed surface 106 or another part of the interconnecting struts 104. In other variations, the growth factors 105 can be injected, incorporated, or otherwise introduced into a hydrogel (e.g., a hyaluronic acid-based hydrogel) and the hydrogel carrying the growth factors can be deposited onto the recessed surface 106.

The growth factors 105 can be released once the interconnecting struts 104 begin to biodegrade after the endovascular scaffold 100 is deployed within a vasculature of the patient. The growth factors 105 can stimulate or facilitate the production of an extracellular matrix (ECM) after the degradation period. For example, the ECM can form at sites formerly occupied by the interconnecting struts 104.

FIG. 1B illustrates the undulating rings 102 of the endovascular scaffold 100 after the plurality of interconnecting struts 104 have biodegraded. As shown in FIG. 1B, discontinuities 108 or gaps/voids can develop in between the undulating rings 102 as the interconnecting struts 104 biodegrade. In some variations, the length of the discontinuities 108 (i.e., the distance between adjacent undulating rings 102) can range from 0.5 mm to 1.0 mm. In other variations, the length of the discontinuities 108 can range from 1.0 mm to 5.0 mm. As shown in FIG. 1B, when the interconnecting struts 104 biodegrade, the undulating rings 102 can be completely separated from one another. In other variations, at least some of the undulating rings 102 can still be connected to one another when some of the interconnecting struts 104 biodegrade.

As previously discussed, localized ECMs can form in spaces or areas formerly occupied by the interconnecting struts 104. In some variations, the interconnecting struts 104 can release growth factors 105 that can stimulate the formation of the ECMs. Moreover, the relatively fast resorption of the interconnecting struts 104 can also elicit an inflammatory response that can cause the ECM to form.

The ECM can comprise proteoglycans, polysaccharides, and proteins. Adhesion proteins within the ECM, including fibronectin and laminin, can be involved in many cellular processes, including tissue repair, embryogenesis, blood clotting, and cell migration/adhesion.

The ECM can form over at least parts of the undulating rings 102. In certain variations, the ECM can form over at least parts of the undulating rings 102 as well as any partially degraded interconnecting struts 104. In this manner, the ECM can strengthen and support what remains of the endovascular scaffold 100 and allow the endovascular scaffold 100 to handle complex loading conditions in peripheral vessels such as the superficial femoral artery (SFA) or the proximal popliteal artery (PPA).

In some variations, at least some of the undulating rings 102 can be coated by one or more layers of anti-inflammatory agents, anti-proliferative agents, or a combination thereof. The anti-inflammatory agents can comprise dexamethasone and/or its derivative. The anti-proliferative agents can comprise sirolimus and/or its derivative. The anti-inflammatory and/or anti-proliferative agents can be incorporated via spray coating on the surface of the endovascular scaffold 100 or mixed into the polymer of the scaffold 100 itself. The anti-inflammatory or anti-proliferative agents can be incorporated at a volume ranging from about 50 μg/cm² to about 250 μg/cm².

FIG. 2 illustrates the endovascular scaffold 100 crimped onto an inflatable balloon 200 of a balloon catheter 202. The endovascular scaffold 100 can be radially compressed into a delivery configuration 204. For example, the undulating rings 102 of the endovascular scaffold 100 can be radially compressed from the initial diameter 114 (see e.g., FIG. 1A) into a delivery diameter 206 (also shown as D2) when the endovascular scaffold 100 is in the delivery configuration 204. The endovascular scaffold 100 can be crimped onto the balloon 200 using a stent crimping machine.

In some variations, the delivery diameter 206 can be between approximately 0.50 mm and 2.00 mm. More specifically, the delivery diameter 206 can be between approximately 0.71 mm and 1.65 mm.

The endovascular scaffold 100 can be crimped onto the inflatable balloon 200 according to a scaffold crimping procedure. The scaffold crimping procedure can comprise crimping the endovascular scaffold 100 onto the balloon 200 in multiple steps at an elevated temperature. In some variations, the endovascular scaffold 100 and the balloon 200 can be heated to temperatures between about 40° C. and 80° C. (e.g., 40° C. to 50° C.) during the crimping process. For example, the endovascular scaffold 100 can be heated to a temperature exceeding the glass transition temperature of the scaffold 100 (e.g., about 70° C.) during the crimping process.

The crimping process can involve reducing the diameter of the endovascular scaffold 100 (more specifically, the undulating rings 102 of the endovascular scaffold 100) gradually in a stepwise manner. Each step of reducing the diameter of the endovascular scaffold 100 can be followed by a pause period. The pause period can range from about 1 second to 60 seconds. The entire crimping process can comprise 5 to 30 steps (with each step followed by the pause period). The total crimping time can range from several seconds to 30 minutes or more.

When the endovascular scaffold 100 is radially compressed into the delivery configuration 204, void spaces 208 or gaps in between the compressed undulating rings 102 can catch portions of the inflatable balloon 200 and allow such portions of the balloon 200 to extend through the void spaces 208 or gaps. For example, multiple portions of the inflatable balloon 200 can extend through these void spaces 208 or gaps along the length of the endovascular scaffold 100. In this manner, the inflatable balloon 200 can stabilize and provide structural support to the endovascular scaffold 100 during delivery and deployment. As such, the inflatable balloon 200 can create a mechanical interlock with the scaffold 100 thereby preventing scaffold 100 from slipping during delivery through the patient's vasculature.

In some variations, the endovascular scaffold 100 can be tapered such that the transition between the balloon 200 and the endovascular scaffold 100 is smooth. Additionally and/or optionally, the scaffold 100 can have edges that can be created using methods including, e.g., angled laser beam, profile machining, control depth profile machining, using laser beam or other mechanisms, injection molding process, abrasive or material removal process, as well as other material removal/deposit processes.

In some variations, the inflatable balloon 200 can be made of polymeric materials with elastic properties. For example, the inflatable balloon 200 can be made from a polyamide (e.g., Pebax®). In other variations, the balloon 200 can be made from materials that include, but are not limited to, polyethylene, nylon-derivatives, and polyolefin copolymers. In other variations, the balloon 200 can be made of elastomeric materials, non-elastomeric materials, or a combination of elastomeric materials and non-elastomeric materials. Elastomeric materials can include elastomeric varieties of latex, silicone, polyurethane, polyolefin elastomers, flexible polyvinyl chloride (PVC), ethylene vinyl acetate (EVA), ethylene methyl acrylate (EMA), ethylene ethyl acrylate (EEA), styrene butadiene styrene (SBS), and ethylene propylene diene rubber (EPDM). Non-elastomeric materials can include polyethylene terephthalate (PET).

The balloon 200 can have a balloon thickness and a balloon length. The balloon thickness can range from about 0.0001 mm to 0.005 mm. The balloon length can range from about 10 mm to 140 mm.

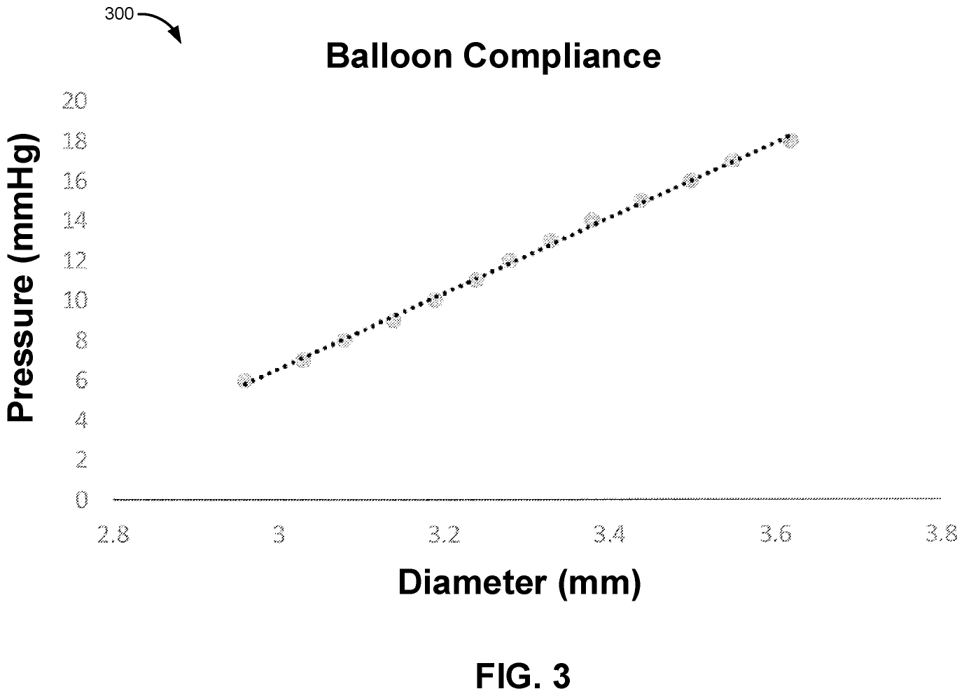

FIG. 3 illustrates a pressure-diameter plot 300 of the inflatable balloon 200 of the balloon catheter 202. The pressure-diameter plot 300 shows a balloon compliance curve of the inflatable balloon 200.

The inflatable balloon 200 can be considered a semi-compliant balloon. A semi-compliant balloon can allow for some degree of balloon expansion in response to an increase in the inflation pressure (although the degree of expansion is usually less than a compliant balloon and more than a non-compliant balloon.

The inflatable balloon 200 can be characterized by an upward sloping compliance curve. As shown in FIG. 3, the inflatable balloon 200 can be expandable to a minimum diameter of about 2.9 mm at about 6.0 ATMs of pressure. The inflatable balloon 200 can also be expandable to a maximum diameter of about 3.7 mm at about 16.0 ATMs of pressure. One unexpected discovery made by the applicant is that a balloon characterized by the compliance curve shown in FIG. 3 is ideal for delivering and deploying the endovascular scaffold 100 disclosed herein.

The amount of radial orientation imparted into the balloon material can contribute to the compliance of the balloon 200. The radial orientation can be controlled by the ratio between a diameter of an extruded tubing and an inner diameter of the balloon mold. In some variations, the ratio between the diameter of an extruded tubing and an inner diameter of the balloon mold can range from 10% to 400%.

As previously described, when the endovascular scaffold 100 is radially compressed into the delivery configuration 204 via the crimping process, portions of the balloon 200 can protrude or extend through the void spaces 208 or gaps in between the undulating rings 102 of the scaffold 100. For example, multiple portions of the inflatable balloon 200 can extend through these void spaces 208 or gaps along the length of the endovascular scaffold 100. This can allow parts of the endovascular scaffold 100 to mechanically interlock, at least temporarily, with parts of the balloon 200. This temporary interlock can help stabilize and provide structural support to the endovascular scaffold 100 during delivery and deployment. Moreover, this temporary interlock can ensure that the undulating rings 102 are properly aligned with one another during delivery and deployment.

FIGS. 4A to 4C illustrate an example method of deploying the endovascular scaffold 100 at a target site 400 within a peripheral vessel 402. Once the endovascular scaffold 100 has been crimped onto the inflatable balloon 200 of the balloon catheter 202, the balloon-scaffold assembly can be placed within a lumen of a delivery catheter 404. The delivery catheter 404 comprising the balloon-scaffold assembly can then be advanced intravascularly through the body of the patient until the delivery catheter 404 reaches the target site 400.

In some variations, a flexible guidewire 406 can be advanced, initially, to the target site 400. The delivery catheter 404 can then be passed over the guidewire 406 to the target site 400. FIG. 4A illustrates that the balloon-scaffold assembly can be exposed upon reaching the target site 400.

FIG. 4B illustrates that the inflatable balloon 200 of the balloon catheter 202 can be inflated to radially expand the endovascular scaffold 100 from the delivery configuration 204 to an expanded configuration 408. The endovascular scaffold 100 can support the peripheral vessel 402 at the target site 400 in the expanded configuration 408.

In some variations, the undulating rings 102 can be radially expanded to the initial diameter 114 (also shown as D1). In other variations, the undulating rings 102 can self-expand from the initial diameter 114 to an even larger deployed diameter.

Once the endovascular scaffold 100 is successfully deployed at the target site 400, a surgeon can deflate the balloon 200 and withdraw the balloon 200 and the delivery catheter 404 from the peripheral vessel 402 of the patient.

FIG. 4C illustrates what remains of the endovascular scaffold 100 once the interconnecting struts 104 have biodegraded. As previously discussed, some or all of the interconnecting struts 104 can biodegrade after approximately 7.0 months. In other variations, some or all of the interconnecting struts 104 can biodegrade between 7.0 months and 2.0 years. In additional variations, some or all of the interconnecting struts 104 can biodegrade after 2.0 years.

As shown in FIG. 4C, discontinuities 108 can develop in between the undulating rings 102 as the interconnecting struts 104 biodegrade. In some variations, when the interconnecting struts 104 biodegrade, the undulating rings 102 can be completely separated from one another.

As previously discussed, the undulating rings 102 can be supported in part by extracellular matrix formed at the discontinuities 108. In some variations, growth factors 105 deposited on the interconnecting struts 104 (e.g., in a recessed surface 106 defined along the interconnecting strut 104) or integrated within one or more layers of the interconnecting struts 104 can elicit formation of the extracellular matrix. The growth factors 105 can comprise at least one of a vascular endothelial growth factor (VEGF), a platelet-derived growth factor (PDGF), and a heparin-binding EGF-like growth factor (HB-EGF).

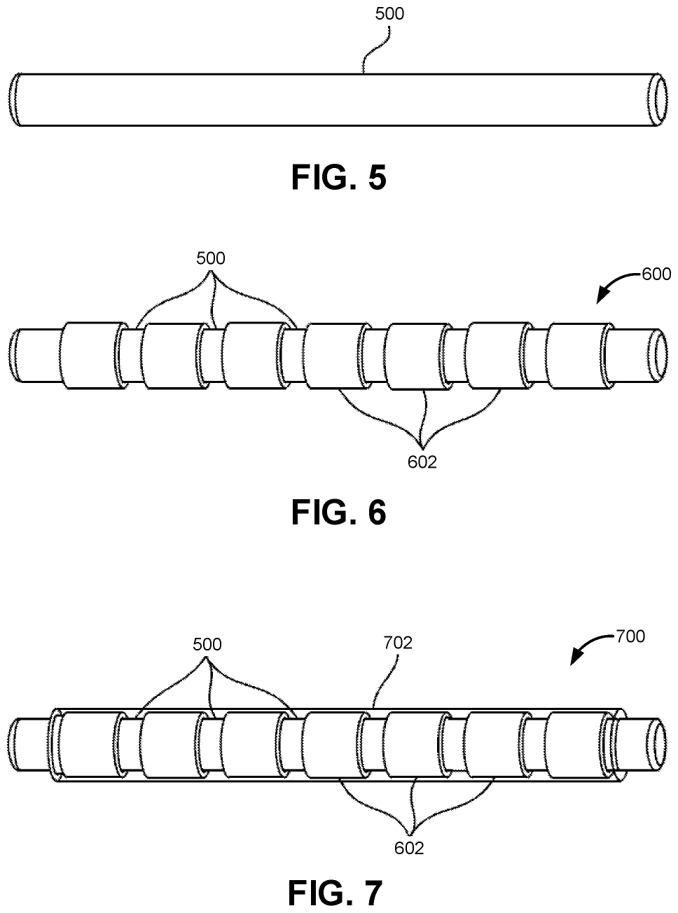

FIG. 5 illustrates an example of a polymeric base substrate 500 that can be used to form components of the endovascular scaffold 100. In some variations, the polymeric base substrate 500 can be used to form the undulating rings 102 of the endovascular scaffold 100. In other variations, the polymeric base substrate 500 can be used to form both the undulating rings 102 and the interconnecting struts 104 of the endovascular scaffold 100.

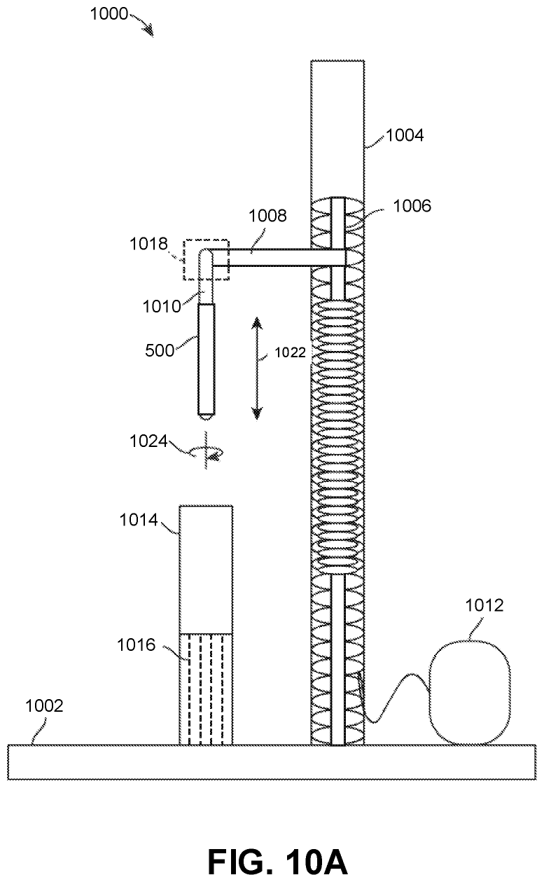

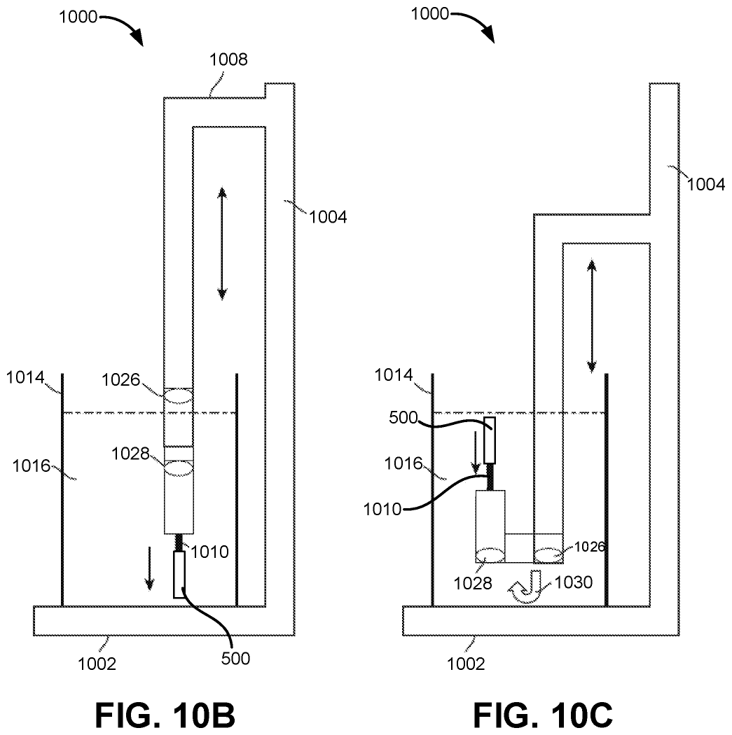

As will be discussed in more detail in later sections, the polymeric base substrate 500 can be an elongate polymeric tube formed via a dip-coating process. The dip-coating process can comprise dipping a mandrel (see, e.g., mandrel 1010 of FIGS. 10A-10C) in a polymeric solution such that a dip-coated polymeric base substrate 500 forms on the mandrel. The undulating rings 102 can then be formed or patterned by laser cutting the dip-coated polymeric base substrate 500. In other variations, the undulating rings 102 can be formed by mechanically machining or cutting the dip-coated polymeric base substrate 500. In further variations, the undulating rings 102 can be formed by subjecting the dip-coated polymeric base substrate 500 to other material removal techniques such as solvent dissolution or abrasive removal.

In some cases, the interconnecting struts 104 can be formed from the same polymeric base substrate 500 using the same pattern-forming techniques (e.g., laser cutting, mechanical machining or cutting, solvent dissolution, abrasive removal, etc.) as the undulating rings 102. For example, when the dip-coated polymeric base substrate 500 is being cut to form the undulating rings 102, additional cuts or patterns can be made to form the interconnecting struts 104. As a more specific example, the interconnecting struts 104 can be formed by cutting away all portions of the dip-coated polymeric base substrate 500 in between adjacent undulating rings 102 other than one or more short strips, bands, or segments connecting such rings 102.

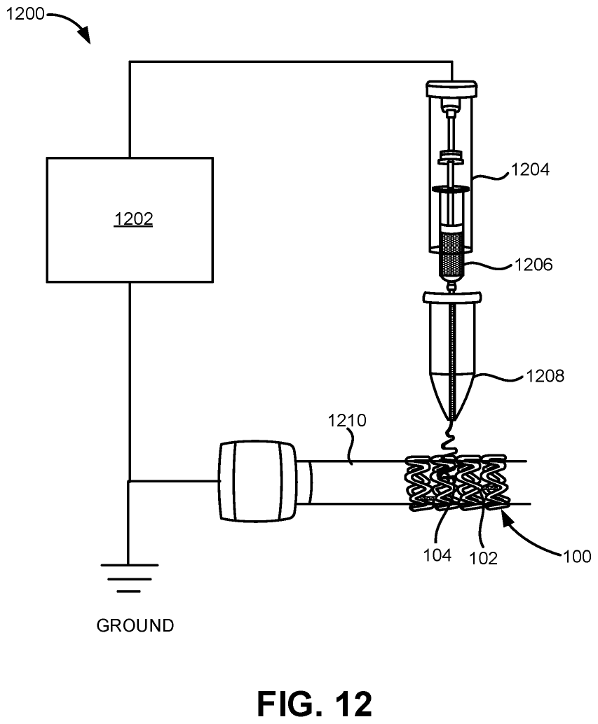

In other variations, only the undulating rings 102 can be formed from one polymeric base substrate 500 and the interconnecting struts 104 can be formed separately using another polymeric base substrate 500 (for example, by cutting another polymeric base substrate 500 into short strips, bands, or segments). In these variations, the interconnecting struts 104 can be adhered, affixed, or otherwise attached to adjacent or neighboring undulating rings 102 to connect such rings 102. In further variations, the interconnecting struts 104 can be formed by electrospinning polymer filaments or strands across adjacent undulating rings 102 (see, e.g., FIG. 12).

The polymeric base substrate 500 can be made of a biodegradable polymeric material such as polylactic acid (PLA) or polylactide. More specifically, the undulating rings 102 can be made of poly-L-lactic acid (PLLA)/poly-L-lactide, poly-DL-lactic acid (PDLLA)/poly-DL-polylactide, or copolymers, terpolymers, combinations, or mixtures thereof.

In other variations, at least part of the polymeric base substrate 500 can be made of poly-glycolic acid (PGA)/poly-glycolide, poly-lactic-co-glycolic acid (PLGA)/poly-lactide-co-glycolide, polycaprolactone such as poly-ε-caprolactone, polydioxanone, polyanhydride, trimethylene carbonate, poly(ß-hydroxybutyrate), poly(g-ethyl glutamate), poly(DTH iminocarbonate), poly(bisphenol A iminocarbonate), poly(ortho ester), polycyanoacrylate, and polyphosphazene, or copolymers, terpolymers, combinations, or mixtures thereof with any of the aforementioned polymers including PLA, PLLA, or PDLLA. In additional variations, at least part of the polymeric base substrate 500 can be made of a biodegradable marine-based polymer such as chitin or chitosan.

FIG. 6 illustrates an example of a multi-layered polymeric base substrate 600 that can be used to form the endovascular scaffold 100. The multi-layered polymeric base substrate 600 can comprise a plurality of base ring structures 602 positioned or fitted upon the polymeric base substrate 500.

In some variations, the base ring structures 602 can be formed by cutting another instance of the polymeric base substrate 600 into multiple ring segments. These ring segments from the other polymeric base substrate 600 can then be fitted, crimped, slid onto, or otherwise positioned on the polymeric base substrate 500.

The portions of the polymeric base substrate 500 covered by the base ring structures 602 can be patterned or otherwise formed into the undulating rings 102. For example, the portions of the polymeric base substrate 500 covered by the base ring structures 602 can be laser cut or mechanically machined or cut into the undulating rings 102. The portions of the polymeric base substrate 500 uncovered by the base ring structures 602 can then be patterned (e.g., laser cut or mechanically machined or cut) or otherwise formed into the interconnecting struts 104. Since the portions of the polymeric base substrate 500 covered by the base ring structures 602 are thicker and comprised of more material than the portions of the polymeric base substrate 500 uncovered by the base ring structures 602, the interconnecting struts 104 made from the latter portions can degrade faster than the undulating rings 102 made from the thicker portions.

FIG. 7 illustrates another example of a multi-layered polymeric base substrate 700 that can be used to form the endovascular scaffold 100. The multi-layered polymeric base substrate 700 can comprise an additional layer 702 overlaid atop the multi-layered polymeric base substrate 600.

In some variations, the additional layer 702 can be a drug-eluting layer. For example, the drug-eluting layer can be a biodegradable polymeric layer having pharmaceutical agents such as anti-inflammatory agents, anti-proliferative agents, and/or anti-restenotic agents incorporated, integrated, or otherwise mixed therein.

In other variations, the additional layer 702 can be a biodegradable polymeric layer having growth factors (e.g., the growth factors 105 shown in FIG. 1A) incorporated, integrated, or otherwise mixed therein. In additional variations, the additional layer 702 can be an elastomeric layer.

Similar to the multi-layered polymeric base substrate 600 shown in FIG. 6, the multi-layered polymeric base substrate 700 can also be laser cut or mechanically machined or cut into the undulating rings 102 and the interconnecting struts 104. For example, the portions of the polymeric base substrate 500 covered by the base ring structures 602 and the additional layer 702 can be laser cut or mechanically machined or cut into the undulating rings 102. The portions of the polymeric base substrate 500 only covered by the additional layer 702 (i.e., uncovered by the base ring structures 602) can then be patterned (e.g., laser cut or mechanically machined or cut) or otherwise formed into the interconnecting struts 104. Since the portions of the polymeric base substrate 500 covered by the base ring structures 602 and the additional layer 702 are thicker and comprised of more material than the portions of the polymeric base substrate 500 only covered by the additional layer 702, the interconnecting struts 104 made from the latter portions can degrade faster than the undulating rings 102 made from the thicker portions.

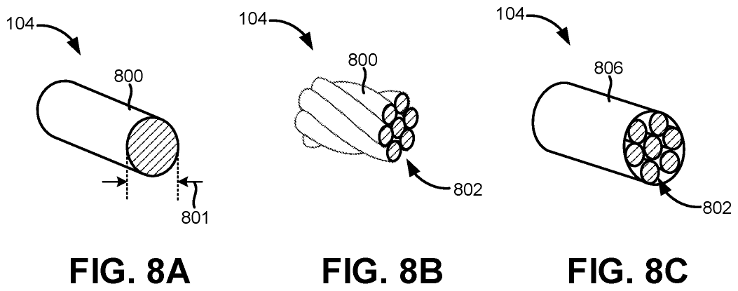

FIGS. 8A-8C illustrate cross-sections of variations of interconnecting struts 104 made of one or more filaments. The filaments can be formed by electrospinning (see e.g., FIG. 12), extrusion, or injection molding.

FIG. 8A illustrates that the interconnecting strut 104 can be made of a monofilament 800. The monofilament 800 can be a single strand or filament formed by electrospinning or extrusion. For example, a monofilament 800 can be laid between adjacent undulating rings 102 to form the interconnecting strut 104. The monofilament 800 can be adhered to or otherwise affixed to the undulating rings 102 by adhesives, welding (e.g., spot welding), or a combination thereof. In some variations, the monofilaments 800 can be embedded into a backbone of a polymer tubing during the deposition process.

The monofilament 800 can have a filament diameter 801. The filament diameter 801 can be between approximately 0.10 mm and 1.0 mm. For example, the filament diameter 801 can be approximately 0.50 mm.

The monofilament 800 can be made of a biodegradable polymeric material. For example, the monofilament 800 can be made of polylactic acid (PLA) or polylactide. More specifically, the monofilament 800 can be made of poly-L-lactic acid (PLLA)/poly-L-lactide, poly-DL-lactic acid (PDLLA)/poly-DL-polylactide, or copolymers, terpolymers, combinations, or mixtures thereof.

In other variations, the monofilament 800 can be made of poly-glycolic acid (PGA)/poly-glycolide, poly-lactic-co-glycolic acid (PLGA)/poly-lactide-co-glycolide, polycaprolactone such as poly-ε-caprolactone, polydioxanone, polyanhydride, trimethylene carbonate, poly(ß-hydroxybutyrate), poly(g-ethyl glutamate), poly(DTH iminocarbonate), poly(bisphenol A iminocarbonate), poly(ortho ester), polycyanoacrylate, and polyphosphazene, or copolymers, terpolymers, combinations, or mixtures thereof with any of the aforementioned polymers including PLA, PLLA, or PDLLA.

In some variations, the monofilament 800 can be made of a biodegradable marine-based polymer. For example, the monofilament 800 can be made of chitin, chitosan, or a combination thereof. In other variations, the monofilament 800 can be made of a chitosan-based composite material reinforced with polymeric fibers.

FIG. 8B illustrates that the interconnecting strut 104 can be made of a plurality of monofilaments 800 assembled into a multifilament 802. In some variations, the multifilament 802 can be formed by braiding or twisting together a number of monofilaments 800. In these variations, the multifilament 802 can be adhered to or otherwise affixed to the undulating rings 102 by adhesives, welding (e.g., spot welding), or a combination thereof.

In other variations, the multifilament 802 can be formed by laying the monofilaments 800 in a biaxial orientation or a multi-axial orientation in between adjacent undulating rings 102. For example, the multifilament 802 can be formed by laying a single monofilament 800 back and forth over adjacent undulating rings 102.

FIG. 8C illustrates that the multifilament 802 can be covered by a coating layer or sheath 806. For example, the multifilament 802 can be dip-coated in a polymeric solution (e.g., a solution of biodegradable polymers) to form the coating layer or sheath 806. The coating or sheath 806 can increase the tensile strength of the interconnecting strut 104 or allow the interconnecting strut 104 to be degraded at a slower rate.

The coating layer or sheath 806 can also be a drug-eluting layer. For example, the drug-eluting layer can be a biodegradable polymeric layer having pharmaceutical agents such as anti-inflammatory agents, anti-proliferative agents, and/or anti-restenotic agents incorporated, integrated, or otherwise mixed therein.

In other variations, the coating layer or sheath 806 can be a biodegradable polymeric layer having growth factors (e.g., the growth factors 105 shown in FIG. 1A) incorporated, integrated, or otherwise mixed therein. In additional variations, the coating layer or sheath 806 can be an elastomeric layer or coating.

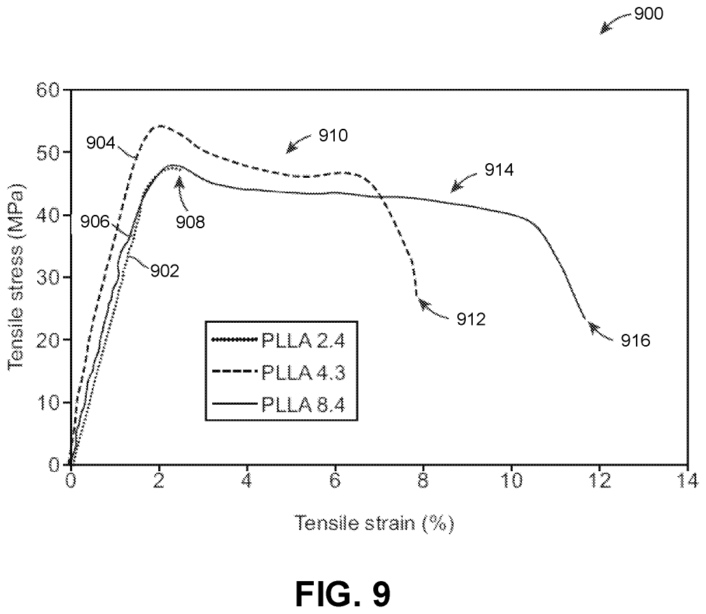

FIG. 9 illustrates a stress-strain plot of poly-L-lactic acid (PLLA) at differing molecular weights and their corresponding stress-strain values indicating brittle fracture to ductile failure. As previously discussed, the undulating rings 102 of the endovascular scaffold 100 can be made in part of a high-molecular weight PLLA. The molecular weight of a polymer is typically one of the factors in determining the mechanical behavior of the polymer. With an increase in the molecular weight of a polymer, there is generally a transition from brittle to ductile failure. An example is illustrated in the stress-strain plot 900 of FIG. 9 which illustrates the differing mechanical behavior resulting from an increase in molecular weight. The stress-strain curve 902 of a sample of PLLA 2.4 shows a failure point 908 having a relatively low tensile strain percentage at a high tensile stress level indicating brittle failure. A sample of PLLA 4.3, which has a relatively higher molecular weight than PLLA 2.4, illustrates a stress-strain curve 904 which has a region of plastic failure 910 after the onset of yielding and a failure point 912 which has a relatively lower tensile stress value at a relatively higher tensile strain percentage indicating a degree of ductility. Yield occurs when a material initially departs from the linearity of a stress-strain curve and experiences an elastic-plastic transition.

A sample of PLLA 8.4, which has yet a higher molecular weight than PLLA 4.3, illustrates a stress-strain curve 906 which has a longer region of plastic failure 914 after the onset of yielding. The failure point 916 also has a relatively lower tensile stress value at a relatively higher tensile strain percentage indicating a degree of ductility.

Thus, a polymeric base substrate 500 can be made using a high-molecular weight PLLA (e.g., PLLA 8.4, PLLA with 8.28 IV, etc.). At least parts of the polymeric base substrate 500 can then be patterned into the undulating rings 102 and the undulating rings 102 can exhibit a stress-strain curve similar to that shown in FIG. 9.

FIG. 10A illustrates an example of a dip-coating assembly 1000 that can be used to form the polymeric base substrate 500 on a mandrel. As previously disclosed, once the polymeric base substrate 500 is formed, the undulating rings 102 (and, in comes cases, the interconnecting struts 104) can be patterned from the polymeric base substrate 500 using techniques known to one of ordinary skill in the art of stent design (e.g., laser cutting, mechanical machining or cutting, solvent dissolution, abrasive tools, etc.).

The utilization of dip-coating to create a polymeric base substrate 500 results in a substrate 500 (and, eventually, an endovascular scaffold 100 from the substrate 500) that is able to retain the inherent beneficial mechanical properties of the starting materials (e.g., PLLA 8.4 or PLLA with 8.28 IV, etc.).

For example, the polymeric base substrate 500 can have high radial strength which is mostly retained through any additional manufacturing processes before the endovascular scaffold 100 is implanted.

The dip-coating process also allows for the creation of polymeric base substrate 500 having multiple layers. The multiple layers can be formed from the same or similar polymeric materials or they can be varied to include any number of additional agents, such as one or more drugs for treatment of the diseased vessel. Moreover, the ability to make a polymeric base substrate 500 with multiple layers can allow one to control other parameters, conditions, or ranges between individual layers such as varying the degradation rate between polymer layers while maintaining the intrinsic molecular weight and mechanical strength of the polymer at a high level with minimal degradation of the starting materials. Furthermore, dip-coating can produce structures having precise geometric tolerances with respect to wall thicknesses, concentricity, diameter, etc.

One technical challenge faced by stent manufacturers is that stents or scaffolds often fail via brittle fracture when placed under stress within the patient's body. It is generally desirable for polymeric stents or scaffolds (such as the endovascular scaffold 100 disclosed herein) to exhibit ductile failure under an applied load rather via brittle failure. This is especially important during delivery and deployment of stents or scaffolds from a constraining sheath and/or using an inflatable balloon catheter. Percent (%) ductility is generally a measure of the degree of plastic deformation that has been sustained by the material at fracture. A material that experiences very little or no plastic deformation upon fracture is brittle.

The dip-coating assembly 1000 can comprise a base 1002 that supports a column 1004 that houses a drive column 1006 and a bracket arm 1008. Motor 1012 can urge drive column 1006 vertically along column 1004 to move bracket arm 1008 accordingly. Mandrel 1010 can be attached to bracket arm 1008 above container 1014 which can be filled with a polymeric solution 1016 (e.g., a solution of any one of the biodegradable polymers previously disclosed) into which mandrel 1010 can be dipped via a linear motion 1022. The one or more polymers can be dissolved in a compatible solvent in one or more corresponding containers 1014 such that the appropriate polymeric solution 1016 can be placed under the mandrel 1010. An optional motor 1018 can be mounted along bracket arm 1008 or elsewhere along assembly 1000 to impart an optional rotational motion 1024 to mandrel 1010 and the polymeric base substrate 500 formed along mandrel 1010 to increase the circumferential strength of the polymeric base substrate 500 during the dip-coating process.

The dip-coating assembly 1000 can be isolated on a vibration-damping or vibrationally isolated table to ensure that the liquid surface held within container 1014 remains completely undisturbed to facilitate the formation of a uniform thickness of polymer material along mandrel 1010 and/or the polymeric base substrate 500 with each deposition. The entire assembly 1000, or just a portion of the assembly such as the mandrel 1010 and the polymeric solution 1016, can be placed in an inert environment such as a nitrogen gas environment while maintaining a very low relative humidity (RH) level, e.g., less than 30% RH, and appropriate dipping temperature, e.g., at least 20° C. below the boiling point of the solvent within container 1014 so as to ensure adequate bonding between polymer layers of the dip-coated polymeric base substrate 500. Multiple mandrels can also be mounted along the bracket arm 1008 or directly to the column 1004.

The mandrel 1010 can be sized appropriately and define a cross-sectional geometry to impart a desired shape or size to the polymeric base substrate 500. The mandrel 1010 can be generally circular in cross-section although geometries can be utilized as desired. In one example, the mandrel 1010 can define a circular geometry having a diameter ranging from 1.0 mm to 20 mm to form a polymeric base substrate 500 having a corresponding inner diameter. Moreover, the mandrel 1010 can be made generally from various materials which are suitable to withstand dip-coating processes, e.g., stainless steel, copper, aluminum, silver, brass, nickel, titanium, etc. The length of the mandrel 1010 that is dipped into the polymeric solution 1016 can be optionally limited in length by, e.g., 50 cm, to ensure that an even coat of polymer is formed along the dipped length of mandrel 1010 to limit the effects of gravity during the coating process. The mandrel 1010 can also be made from a polymeric material that is lubricious, strong, has good dimensional stability, and is chemically resistant to the polymeric solution utilized for dip-coating.

Moreover, the mandrel 1010 can have a smooth surface for the polymeric solution 1016 to form upon. In other variations, the mandrel 1010 can define a surface that is coated with a material such as polytetrafluorethylene to enhance removal of the polymeric base substrate formed thereon. In yet other variations, the mandrel 1010 can be configured to define any number of patterns over the surface of the mandrel 1010, e.g., either over the entire length of the mandrel 1010 or just a portion of the surface of the mandrel 1010.

In some variations, surface patterns can be mold-transferred during the dip-coating process to a surface of the polymeric base substrate 500. The mold-transferred pattern can form raised or depressed sections and can include undulating patterns, checkered patterns, cross-hatched patterns, cratered patterns, etc. that can enhance endothelialization with the surrounding tissue soon after the endovascular scaffold 100 is implanted within a patient.

The dipping direction can be alternated or changed between layers of the polymeric base substrate 500. In some variations, a previously dipped polymeric base substrate 500 can be removed from the mandrel 1010 and replaced onto the mandrel 1010 in an opposite direction before the dipping-coating process is continued. Alternatively, the mandrel 1010 can be angled relative to the bracket arm 1008 and/or the polymeric solution 1016 during or prior to the dipping-coating process.

FIGS. 10B and 10C illustrate additional examples of a dip-coating assembly 1000 comprising one or more articulatable linkages (e.g., articulable linkages 1026 and 1028) that can adjust a dipping direction of the mandrel 1010. For example, the dip-coating assemblies 1000 depicted in FIGS. 10B and 10C can achieve a uniform polymer wall thickness throughout the length of the formed polymeric base substrate 500 after each dipping step. For instance, after one to three coats are formed in a first dipping direction, additional layers formed upon the initial layers can be formed by dipping mandrel 1010 in a second direction opposite to the first dipping direction, e.g., angling the mandrel 1010 anywhere up to 180° from the first dipping direction. This can be accomplished, in one example, using pivoting linkage 1026 and/or pivoting linkage 1028. The one or more pivoting linkages (e.g., pivoting linkage 1026 and/or pivoting linkage 1028) can maintain the mandrel 1010 in a first vertical position relative to the polymeric solution 1016 to coat the initial layers of the polymeric base substrate 500. The one or more pivoting linkages (e.g., pivoting linkage 1026 and/or pivoting linkage 1028) can then be actuated to reconfigure the mandrel 1010 from its first vertical position to a second vertical position opposite to the first vertical position, as indicated by direction 1030 (see, e.g., FIG. 10C). With repositioning of the mandrel 1010 complete, the dipping process can be resumed by dipping the entire linkage assembly along with the mandrel 1010 and the polymeric base substrate 500. In this manner, neither the mandrel 1010 nor the polymeric base substrate 500 needs to be removed, thus eliminating any risk of contamination. The one or more pivoting linkages (e.g., pivoting linkage 1026 and/or pivoting linkage 1028) can comprise any number of mechanical or electromechanical pivoting and/or rotating mechanisms know to those of ordinary skill in the art.

Dipping the mandrel 1010 and the polymeric base substrate 500 in different directions can also enable the coated layers to have a uniform thickness throughout from its proximal end to its distal end to help compensate for the effects of gravity during the coating process. These values are intended to be illustrative and are not intended to be limiting in any manner. Any excess dip-coated layers on the one or more pivoting linkages (e.g., pivoting linkage 1026 and/or pivoting linkage 1028) can simply be removed from the mandrel 1010 by breaking the layers. Alternating the dipping direction can also result in the polymers being oriented alternately which can reinforce the tensile strength in the axial direction of the dip-coated polymeric base substrate 500.

The polymeric base substrate 500 can also be any of the dip-coated substrates disclosed in U.S. patent application Ser. Nos. 12/541,095 and 14/030,912, the contents of which are hereby incorporated by reference in their entireties. Since the polymeric base substrate 500 can be formed to have one or more layers overlaid upon one another, the polymeric base substrate 500 can be formed to have a first layer of a first polymer, a second layer of a second polymer, and so on depending upon the desired structure and properties of the substrate. Thus, the various polymeric solutions 1016 and containers 1014 can be replaced beneath the mandrel 1010 between dip-coating steps in accordance with the desired layers to be formed upon the substrate such that the mandrel 1010 can be dipped sequentially into the appropriate polymeric solution.

Depending upon the desired wall thickness of the formed polymeric base substrate 500, the mandrel 1010 can be dipped into the appropriate solution as determined by the number of times the mandrel 1010 is immersed, the duration of time of each immersion within the solution, as well as the delay time between each immersion or the drying or curing time between dips. Additionally, parameters such as the dipping and/or withdrawal rate of the mandrel 1010 from the polymeric solution 1016 can also be controlled to range from, e.g., 5 mm/min to 1000 mm/min. Formation via the dip-coating process can result in a polymeric base substrate 500 having half the wall thickness while retaining an increased level of strength in the polymeric base substrate 500 as compared to an extruded polymeric structure. For example, to form a polymeric base substrate 500 having a wall thickness of, e.g., 200 μm, built up of multiple layers of polylactic acid, the mandrel 1010 can be dipped between, e.g., 2 to 20 times or more, into the polymeric solution 1016 with an immersion time ranging from, e.g., 15 seconds (or less) to 240 minutes (or more). Moreover, the polymeric base substrate 500 and the mandrel 1010 can be optionally dried or cured for a period of time ranging from, e.g., 15 seconds (or less) to 60 minutes (or more) between each immersion. These values are intended to be illustrative and are not intended to be limiting in any manner.

Aside from utilizing polymeric materials having a relatively high molecular weight, another parameter that can be considered to further increase the ductility of the material is its crystallinity. Crystallinity refers to the degree of structural order in the underlying polymeric material. Such polymeric materials can contain a mixture of crystalline and amorphous regions where reducing the percentage of the crystalline regions in the polymer can further increase the ductility of the material. Polymeric materials not only having a relatively high molecular weight but also having a relatively low crystalline percentage can be utilized in the processes described herein to form a desirable tubular substrate.

The following Table 1 shows examples of various polymeric materials (e.g., PLLA IV 8.28 and PDLLA 96/4) to illustrate the molecular weights of the materials in comparison to their respective crystallinity percentage. The glass transition temperature, T_{g}, as well as melting temperature, Tₘ, are given as well. An example of PLLA IV 8.28 is shown illustrating the raw resin and tube-shaped polymeric base substrate 500 as having the same molecular weight, M_{w}, of 1.70×10⁶ gram/mol. However, the crystallinity percentage of PLLA IV 8.28 Resin is 61.90% while the corresponding tube-shaped polymeric base substrate 500 is 38.40%. Similarly, for PDLLA 96/4, the resin form and tube-shaped polymeric base substrate 500 can each have a molecular weight, M_{w}, of 9.80×10⁵ gram/mol; however, the crystallinity percentages are 46.20% and 20.90%, respectively.

TABLE 1
Various polymeric materials and their
respective crystallinity percentages.

| | | | Crystallinity | M_{w} |
|---|---|---|---|---|
| Material | T_{g} (° C.) | Tₘ (° C.) | (%) | (gram/mol) |
| PLLA IV8.28 Resin | 72.5 | 186.4 | 61.90% | 1.70 × 10⁶ |
| PLLA IV8.28 Tubes | 73.3 | 176.3 | 38.40% | 1.70 × 10⁶ |
| PDLLA 96/4 Resin | 61.8 | 155.9 | 46.20% | 9.80 × 10⁵ |
| PDLLA 96/4 Tubes | 60.3 | 146.9 | 20.90% | 9.80 × 10⁵ |

As the resin is dip coated to form the polymeric base substrate 500 using the methods described herein, drying procedures can help preserve the relatively high molecular weight of the polymer from the starting material and throughout processing to substrate and stent formation. Moreover, the drying processes, in particular, can facilitate the formation of desirable crystallinity percentages, as described above.

Aside from the crystallinity of the materials, the immersion times, as well as drying times, can be uniform between each immersion or they can be varied as determined by the desired properties of the resulting substrate. Moreover, the polymeric base substrate 500 can be placed in an oven or dried at ambient temperature between each immersion or after the final immersion to attain a predetermined level of crystals, e.g., 60%, and a level of amorphous polymeric structure, e.g., 40%. Each of the layers overlaid upon one another during the dip-coating process can tightly adhere to one another and the mechanical properties of each polymer are retained in their respective layer with no limitation on the molecular weight of the polymers utilized.

Varying the drying conditions of the materials can also be controlled to affect desirable material parameters. The polymers can be dried at or above the glass transition temperature (e.g., 10° to 20° C. above the glass transition temperature, T_{g}) of the respective polymer to effectively remove any residual solvents from the polymers to attain residual levels of less than 100 ppm, e.g., between 20 to 100 ppm. The polymeric base substrate 500 can also be positioned in a particular manner during the drying step to further enhance the mechanical properties of the polymeric base substrate 500. For instance, the polymeric base substrate 500 can be maintained in a perpendicular drying position (i.e., perpendicular relative to the ground) to maintain the concentricity of the tubular-shaped polymeric base substrate 500.

The polymeric base substrate 500 can be dried in an oven at or above the glass transition temperature of the biodegradable polymers for a period of time ranging anywhere from, e.g., 10 days to 30 days or more.

Additionally, and/or optionally, a shape memory effect can be induced in the polymer during drying of the polymeric base substrate 500. For instance, a shape memory effect can be induced in a tubular-shaped polymeric base substrate 500 to set the tubular shape at the diameter that was formed during the dip-coating process. An example of this is to form a tubular-shaped polymeric base substrate 500 by the dip-coating process described herein at an outer diameter of 5 mm and subjecting the polymeric base substrate 500 to temperatures above the polymer's glass transition temperature, T_{g}. At its elevated temperature, the polymeric base substrate 500 can be elongated, e.g., from a length of 5 cm to 7 cm, while the outer diameter of the polymeric base substrate 500 can be reduced from 5 mm to 3 mm. Of course, these examples are merely illustrative.

Once lengthened and reduced in diameter, the polymeric base substrate 500 can be quenched or cooled in temperature to a sub-T_{g} temperature, e.g., about 20° C. below its T_{g}, to allow the polymeric base substrate 500 to transition back to its glass state. This effectively imparts a shape memory effect of self-expansion to the original diameter of the polymeric base substrate 500. When such a polymeric base substrate 500 is compressed or expanded to a smaller or larger diameter and later exposed to an elevated temperature, the polymeric base substrate 500 can revert to its original 5 mm diameter. This post-processing can also be useful for enabling self-expansion of the polymeric base substrate 500 after a process like laser cutting where the polymeric base substrate 500 is typically heated to its glass transition temperature, T_{g}.

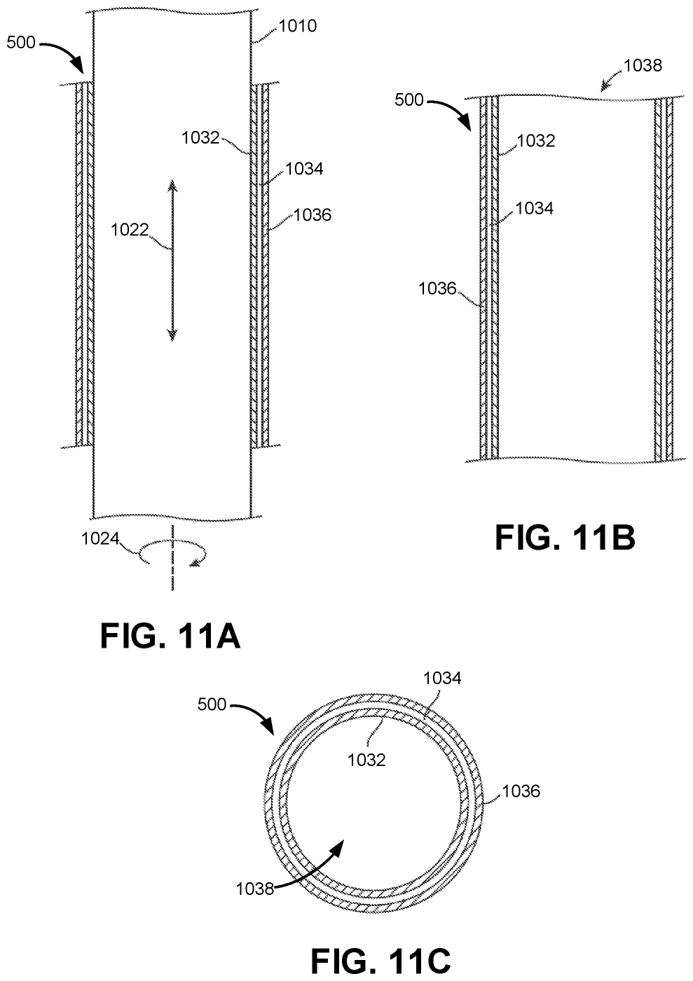

FIGS. 11A to 11B illustrate partial cross-sectional side views of a variation of a multi-layered polymeric base substrate 500 formed along the mandrel 1010. The multi-layered polymeric base substrate 500 can be formed along the mandrel 1010 to have a first layer 1032 formed of a first polymer (e.g., poly-L-lactide). After the formation of the first layer 1032, an optional second layer 1034 of polymer (e.g., poly-L-lactide-co-glycolide), can be formed upon the first layer 1032. Yet another optional third layer 1036 of polymer (e.g., poly-d,l-lactide-co-glycolide) can be formed upon the second layer 1034 to form a resulting substrate defining a lumen 1038 therethrough. One or more of the layers can be formed to degrade at a specified rate or to elute any number of drugs or agents.

FIG. 11C illustrates a cross-sectional end view of the multi-layered polymeric base substrate 500 shown in FIG. 11A. The multilayered polymeric base substrate 500 can comprise three polymeric layers (e.g., layers 1032, 1034, and 1036). In this example, the first layer 1032 can have a molecular weight of Mₙ₁, the second layer 1034 can have a molecular weight of Mₙ₂, and the third layer 1036 can have a molecular weight of Mₙ₃. The endovascular scaffold 100 formed from the polymeric base substrate 500 can be formed in such a manner that the relative molecular weights are Mₙ₁>Mₙ₂>Mₙ₃. This can result in an endovascular scaffold 100 where the layers preferentially degrade at different rates/times beginning with the inner first layer 1032 and eventually degrading to the middle second layer 1034 and finally to the outer third layer 1036 when deployed within the patient's body. Alternatively, the endovascular scaffold 100 can be fabricated where the relative molecular weights are Mₙ₁<Mₙ₂<Mₙ₃. This can result in an endovascular scaffold 100 where the layers preferentially degrade at different rates/times beginning with the outer third layer 1036 and degrading towards the inner first layer 1032. This example is intended to be illustrative and fewer than or more than three layers can be utilized in other examples. Additionally, the molecular weights of each respective layer can be altered in other examples to vary the degradation rates along different layers, if so desired.

Moreover, any one or more of the layers can be formed to impart specified mechanical properties to the polymeric base substrate 500 such that the composite mechanical properties of the resulting polymeric base substrate 500 can specifically be tuned or designed. Additionally, although three layers are illustrated in this example, any number of layers can be utilized depending upon the desired mechanical properties of the polymeric base substrate 500.

Moreover, as multiple layers are overlaid on top of one another in forming the polymeric base substrate 500, certain layers can be designated for a particular function. For example, one or more layers can be designed as load-bearing layers to provide structural integrity to the endovascular scaffold 100 while certain other layers can be allocated for drug-loading or eluting. Those layers which are designated for structural support can be formed from high-molecular weight polymers, e.g., PLLA or any other suitable polymer described herein, to provide a high degree of strength by omitting any drugs as certain pharmaceutical agents can adversely affect the mechanical properties of polymers. Those layers which are designated for drug-loading can be placed within, upon, or between the structural layers.

Additionally, multiple layers of different drugs can be loaded within the various layers. The manner and rate of drug release from multiple layers can depend in part upon the degradation rates of the substrate materials. For instance, polymers that degrade relatively quickly can release their drugs layer-by-layer as each successive layer degrades to expose the next underlying layer. In other variations, drug release can typically occur from a multilayer matrix via a combination of diffusion and degradation. In one example, a first layer can elute a first drug for, e.g., the first 30 to 40 days after implantation. Once the first layer has been exhausted or degraded, a second underlying layer having a second drug can release this drug for the next 30 to 40 days, and so on if so desired. In the example of FIG. 11C, for a stent or scaffold manufactured from the polymeric base substrate 500, outer layer 1036 can contain a first drug for release while the middle layer 1034 can contain a second drug for release after exhaustion or degradation of the outer layer 1036. The underlying inner layer 1032 can provide uncompromised structural support to the entire structure.

In other examples, rather than having each successive layer elute its respective drug, each layer can elute its respective drug simultaneously or at differing rates via a combination of diffusion and degradation. Although three layers are illustrated in this example, any number of layers can be utilized with any practicable combination of drugs for delivery. Moreover, the release kinetics of each drug from each layer can be altered in a variety of ways by changing the formulation of the drug-containing layer.

Examples of drugs or agents that can be loaded within certain layers of the polymeric base substrate 500 can include one or more antiproliferative, antineoplastic, antigenic, anti-inflammatory, and/or anti-restenotic agents. The therapeutic agents can also include anti-lipids, metalloproteinase inhibitors, anti-sclerosing agents. Therapeutic agents can also include peptides, enzymes, radioisotopes or agents for a variety of treatment options. This list of drugs or agents is presented to be illustrative and is not intended to be limiting.

Similarly, certain other layers can be loaded with radio-opaque substances such as platinum, gold, etc. to enable visibility of the stent under imaging modalities such as fluoroscopic imaging. Radio-opaque substances like tungsten, platinum, gold, etc. can be mixed with the polymeric solution and dip-coated upon the substrate such that the radio-opaque substances form a thin sub-micron thick layer upon the substrate. The radio-opaque substances can thus become embedded within layers that degrade in the final stages of degradation or within the structural layers to facilitate stent visibility under an imaging modality, such as fluoroscopy, throughout the life of the implanted device before fully degrading or losing its mechanical strength. Radio-opaque marker layers can also be dip-coated at one or both ends of polymeric base substrate 500, e.g., up to 0.5 mm from each respective end.

Additionally, the radio-opaque substances can also be spray-coated or cast along a portion of the polymeric base substrate 500 between its proximal and distal ends in a radial direction. Rings of polymers having radio-opaque markers can also be formed as part of the structure of the polymeric base substrate 500.

FIG. 12 illustrates an example of an electrospinning assembly 1200 which can be utilized to form the interconnecting struts 104 or part of the undulating rings 102. In some variations, the electrospinning assembly 1200 can be used to form the interconnecting struts 104 after the undulating rings 102 are formed from a dip-coated polymeric base substrate 500.

The electrospinning assembly 1200 can comprise a power supply 1202, a syringe 1204 housing the polymeric solution 1206, a spinneret 1208, and a mandrel 1210 electrically coupled to the power supply 1202. In some variations, the undulating rings 102 formed from the dip-coated polymeric base substrate 500 can be placed on the mandrel 1210 during the electrospinning process.

The syringe 1204 can be electrically coupled to the power supply 1202. The power supply 1202 can supply voltage to the syringe 1204 for the electrospinning process.

The syringe 1204 can include a syringe pump, syringe barrel, and syringe needle. The polymeric solution 1206 can be contained within the syringe barrel. In other variations, the polymeric solution 1206 can be introduced via conduits or tubes into the syringe 1204.

The polymeric solution 1206 can be injected by the syringe 1204 into the spinneret 1208. The spinneret 1208 can be electrically charged. The target (i.e., the mandrel 1210 and/or the undulating rings 102 on the mandrel 1210) can have an opposing electrical charge to the spinneret 1208. The charges can be provided by the power supply 1202 which can create a first charge at the spinneret 1208 and an opposing charge at the target. The polymer can be electrostatically charged via contact with the charged spinneret 1208. The electrostatically charged polymer can then be spun onto the mandrel 1210 and/or the undulating rings 102.

During the electrospinning process, the mandrel 1210 can be rotated and/or translated axially to form interconnecting struts 104 along the length of the endovascular scaffold 100 and around the circumference of the endovascular scaffold 100. The speed at which the mandrel 1210 is rotated or translated can be adjusted to change certain properties of the interconnecting struts 104. In some variations, the density of the interconnecting struts 104 can be influenced by the speed at which mandrel 1210 is rotated or translated. The rotational speed of mandrel 1210 can also influence parameters such as the orientation of the electrospun materials.

The polymeric solution 1206 can be the polymeric solution disclosed previously.

Alternatively, the electrospinning process can be used to make at least part of undulating rings 102 of scaffold 100. The undulating rings 102 can be made using a similar electrospinning process as the interconnecting struts 104. Likewise, during the electrospinning process, varying the rotation or translation speed of the mandrel 1210 can have an effect on the mechanical properties of the undulating rings 102.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps operations can be provided, or steps or operations can be eliminated or omitted from the described methods or processes to achieve the desired results. Moreover, any components or parts of any apparatus or systems described in this disclosure or depicted in the figures can be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings can be regarded in an illustrative rather than a restrictive sense.

Each of the individual variations or embodiments described and illustrated herein has discrete components and features which can be readily separated from or combined with the features of any of the other variations or embodiments. Modifications can be made to adapt a particular situation, material, composition of matter, process, process act(s) or step(s) to the objective(s), spirit or scope of the present invention.

Methods recited herein can be carried out in any order of the recited events that is logically possible, as well as the recited order of events. Moreover, additional steps or operations can be provided or steps or operations can be eliminated to achieve the desired result.

Furthermore, where a range of values is provided, every intervening value between the upper and lower limit of that range and any other stated or intervening value in that stated range is encompassed within the invention. Also, any optional feature of the inventive variations described can be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter can conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of”, when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved. Finally, terms of degree such as “substantially”, “about” and “approximately” as used herein mean a reasonable amount of deviation (e.g., a deviation of up to ±0.1%, ±1%, ±5%, or ±10%, as such variations are appropriate) from the specified value such that the end result is not significantly or materially changed.

Finally, terms of degree such as “substantially”, “about” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about/approximately 1.0 m” can be interpreted to mean “1.0 m” or between “0.9 m and 1.1 m.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that can become obvious to those skilled in the art in view of this disclosure.

1. An endovascular scaffold for use in a peripheral vessel, comprising:
a plurality of undulating rings; and
a plurality of interconnecting struts connecting the plurality of undulating rings to one another,
wherein the plurality of undulating rings are radially compressible into a delivery configuration and expandable from the delivery configuration to an expanded configuration when deployed, and
wherein at least some of the interconnecting struts are configured to biodegrade over a degradation period after the endovascular scaffold is deployed within the peripheral vessel.

2. The endovascular scaffold of claim 1, wherein at least one of the plurality of interconnecting struts comprises growth factors disposed thereon or integrated therein.

3. The endovascular scaffold of claim 2, wherein the growth factors comprise at least one of a vascular endothelial growth factor (VEGF), a platelet-derived growth factor (PDGF), and a heparin-binding EGF-like growth factor (HB-EGF).

4. The endovascular scaffold of claim 2, wherein at least one of the plurality of interconnecting struts comprises a recessed surface defined along the at least one interconnecting strut, and wherein the recessed surface is configured to contain at least some of the growth factors.

5. The endovascular scaffold of claim 1, wherein at least one of the plurality of interconnecting struts is made in part of chitin, chitosan, or a combination thereof.

6. The endovascular scaffold of claim 1, wherein the degradation period is between about 7 months and 24 months.

7. The endovascular scaffold of claim 1, wherein each of the plurality of interconnecting struts is positioned between adjacent undulating rings.

8. The endovascular scaffold of claim 1, wherein at least one of the plurality of interconnecting struts has a width which is less than a circumference of at least one of the plurality of undulating rings.

9. The endovascular scaffold of claim 1, wherein the plurality of undulating rings are biodegradable.

10. The endovascular scaffold of claim 9, wherein the plurality of undulating rings are configured to biodegrade at a slower rate than the plurality of interconnecting struts.

11. The endovascular scaffold of claim 10, wherein the plurality of undulating rings are configured to biodegrade between about 3.0 years and 10.0 years after deployment within the peripheral vessel.

12. The endovascular scaffold of claim 1, wherein the undulating rings deployed within the peripheral vessel are supported in part by an extracellular matrix formed at discontinuities developed in between the undulating rings as the interconnecting struts biodegrade.

13. The endovascular scaffold of claim 1, wherein at least one of the plurality of interconnecting struts is formed by electrospinning.

14. The endovascular scaffold of claim 1, wherein at least one of the plurality of interconnecting struts is comprised of a monofilament.

15. The endovascular scaffold of claim 1, wherein at least one of the plurality of interconnecting struts is comprised of a plurality of filaments in a multifilament configuration.

16. A balloon-scaffold assembly for use in a peripheral vessel, comprising:
an inflatable balloon of a balloon catheter, wherein the inflatable balloon is expandable to a minimum diameter of about 2.9 mm at about 6.0 ATMs of pressure and expandable to a maximum diameter of about 3.7 mm at about 16.0 ATMs of pressure, wherein the inflatable balloon is also characterized by an upward sloping compliance curve; and
an endovascular scaffold crimped onto the inflatable balloon in a delivery configuration, wherein the endovascular scaffold comprises:
a plurality of undulating rings, and
a plurality of interconnecting struts connecting the plurality of undulating rings to one another,
wherein portions of the inflatable balloon extend through void spaces in between the plurality of undulating rings when the plurality of undulating rings are radially compressed into the delivery configuration, and
wherein the inflatable balloon provides structural support to the endovascular scaffold during delivery.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. A method of supporting a peripheral vessel using a balloon-scaffold assembly, the method comprising:
introducing a flexible guidewire to a target site within the peripheral vessel;
advancing a delivery catheter comprising a balloon-scaffold assembly over the guidewire to the target site;
exposing the balloon-scaffold assembly at the target site, wherein the balloon-scaffold assembly comprises:
a balloon of a balloon catheter, and
an endovascular scaffold crimped onto the balloon in a delivery configuration, wherein the endovascular scaffold comprises:
a plurality of undulating rings, and
a plurality of interconnecting struts connecting the plurality of undulating rings to one another,
wherein at least some of the interconnecting struts are configured to biodegrade over a degradation period after deployment,
wherein portions of the balloon extend through void spaces in between the plurality of undulating rings when the plurality of undulating rings are radially compressed into the delivery configuration, and
wherein the balloon provides structural support to the endovascular scaffold during delivery;
inflating the balloon to radially expand the endovascular scaffold to an expanded configuration at the target site;
deflating the balloon; and
withdrawing the delivery catheter and the balloon from the peripheral vessel, wherein the endovascular scaffold in the expanded configuration provides support for the peripheral vessel at the target site.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. A method of making an endovascular scaffold for use in a peripheral vessel, the method comprising:
dipping a mandrel in a polymeric solution such that a dip-coated substrate forms on the mandrel;
forming a plurality of undulating rings from the dip-coated substrate, wherein the plurality of undulating rings are radially compressible into a delivery configuration and expandable from the delivery configuration to an expanded configuration when deployed; and
forming a plurality of interconnecting struts connecting the plurality of undulating rings, wherein at least some of the interconnecting struts are configured to biodegrade over a degradation period when the endovascular scaffold is deployed within the peripheral vessel.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)
